(19) United States
(12) Patent Application Publication
(10) Pub. No.: US 2017/0014640 A1
(43) Pub. Date: Jan. 19, 2017

(54) Portable, HANDS-FREE, PRE-CALIBRATED and WEARABLE Laser brace/wrap type clinical-strength medical device/apparatus, with embedded Low-Level-Laser-Therapy (LLLT), providing for a new method/modality for Pain relief in the form of orthopedic LASERWRAPS, from joint related musculoskeletal pain caused by joint related illnesses including - TENNIS ELBOW, CARPEL-TUNNEL, ARTHRITIS, OSTEOPEROSIS, PLANTAR FASCITIS, TENDONITIS (BACK PAIN, KNEE TENDONITIS, HAND TENDONITIS, ACHILLES TENDONITIS), SPORT INJURIES & BURSITIS

(71) Applicant: Abijith Kariguddaiah, Danville, CA (US)

(72) Inventor: Abijith Kariguddaiah, Danville, CA (US)

(21) Appl. No.: 14/583,792

(22) Filed: Dec. 29, 2014

(51) Int. Cl. A61N 5/06; A61F 5/01; A61F 5/02

(52) U.S. Cl. CPC A61N 5/0613; A61F 5/028; A61F 5/0109; A61F 5/0111; A61F 5/0118; A61N 2005/067

(57) ABSTRACT

WEARABLE, PRE-CALIBRATED & HANDS-FREE CLINICAL-STRENGTH medical device/s for JOINT PAIN RELIEF using a sports/orthopedic brace/wrap (for Wrist, Knee, Elbow, Neck & Shoulder, Ankle & Foot & Back) embedded with therapeutic Low Level Laser therapy (LLLT) based red light (infrared and near infrared) technology. —A convenient PORTABLE device for pain relief from joint related musculoskeletal pain caused by illnesses including—TENDONITIS (BACK PAIN, KNEE TENDONITIS, HAND TENDONITIS, ACHILLES TENDONITIS) TENNIS ELBOW, CARPEL-TUNNEL, ARTHRITIS, OSTEOPEROSIS, PLANTAR FASCITIS, TISSUE HEALING from SPORTS INJURIES AND BURSITIS, and as an non-invasive alternative to NSAIDs (Non-Steroidal Anti Inflammatory Drugs)

### FIELD OF THE INVENTION

This disclosure relates generally to the field of medical instruments, and, in one embodiment, to several methods, a system and several systems, and apparatus of a Low Level laser technology (LLLT) based clinical strength medical instrument for providing PAIN SYMPTOM relief from joint related illnesses including Tendonitis (Back Pain, Knee Tendonitis, Hand Tendonitis, Achilles Tendonitis) Tennis Elbow, Carpel-Tunnel, Arthritis, Osteoporosis, Plantar Fasciitis, Tissue Healing From Sports Injuries And Bursitis. Also relates to the relatively new mobile digital health technologies intended to bring self-care and improve patient lives.

### BACKGROUND OF THE INVENTION

Arthritis & Osteoporosis are most common joint pain diseases that affect millions of people each year. A patient may suffer from arthritis in a variety of locations on the body of a patient. Arthritis may impair a daily routine of the patient. Such impairment can affect the ability of a patient to work or enjoy leisurely activities. Treatments for the pain associated with arthritis include medications and creams. Symptoms of arthritis include pain, swelling, inflammation, limited range of motion, and redness. Medications for treating the pain include acetaminophen, (e.g., Tylenol®), NSAIDS—non-steroidal anti-inflammatory drugs (e.g., Advil®, Motrin®, Aleve®), and tramadol.

Use of acetaminophen can cause liver damage, especially if the patient consumes alcohol. Acetaminophen may also affect the efficacy of other medications taken by the patient. Side-effects of non-steroidal anti-inflammatory drugs include gastric ulcers, cardiovascular problems, and gastrointestinal bleeding. Tramadol may cause nausea and constipation. A patient may try creams as an alternative to, or in addition to, medications. Side effects of creams include irritation or redness on the application site of the cream. Both medications and creams have undesirable side-effects. (Source#1—http://www.medicalnewstoday.com/articles/46607.php)

An Introduction to LLLT (Low Level Laser Therapy)—

LLLT a non-invasive treatment, has been used for many years to relieve pain from sports injuries, joint related arthritic illnesses, neck and back pain, shoulder, wrist, knee and ankle related joint pains.

Low levels of laser energy have a non-thermal, biostimulative effect on biological tissues. The therapeutic application of low level laser energy, frequently known as low level laser therapy (LLLT), produces beneficial clinical effects in the treatment of musculoskeletal, neurological and soft tissue conditions. LLLT is non-invasive and avoids the potential side effects of drug therapy. More specifically, LLLT delivers photons to targeted tissue, penetrating the layers of skin to reach internal tissues to produce a specific, non thermal photochemical effect at the cellular level. Jeffrey R. Basford, Laser Therapy: Scientific Basis and Clinical Role, ORTHOPEDICS, May 1993, at 541.

Low level laser therapy (LLLT) is the application of light (usually a low power laser or LED/VCSEL in the range of 10 mW-500 mW power) to a pathology to promote pain-relief, reduce inflammation and tissue regeneration. The light is typically of narrow spectral width in the red or near infrared (NIR) spectrum (600 nm-900 nm), with a power density (irradiance) between 1 mW-500 mW/cm2. It is typically applied to the injury for a minute or so, a few times a week for several weeks. Unlike other medical laser procedures, LLLT is not an ablative or thermal mechanism, but rather a photochemical effect comparable to photosynthesis in plants whereby the light is absorbed and exerts a chemical change.

(Source: #2 Dose-Response (Prepress) Formerly Nonlinearity in Biology, Toxicology, and Medicine Copyright © 2009 University of Massachusetts)

Low level laser therapy apparatus U.S. Pat. No. 6,312,451B1—Low level laser therapy apparatus for treatment of various tissue injuries is described. The apparatus is described and includes a handheld laser probe coupled to a control unit for selecting and controlling laser energy dosage from about 1 joule/point to about 10 joules/point. The apparatus is described to emit laser energy at a wavelength from about 630 nm to about 904 nm, with a mean power output of between about 100 mW to about 500 mW. The apparatus also describes to include an access control mechanism to limit operability to trained personnel. (Source #7—U.S. Pat. No. 6,312,451 B1 Low level laser therapy treatment apparatus for various tissue injuries)

LLLT is Therapeutic in 2 Ways for Pain Relief:

1. Anti-inflammatory. LLLT reduces oxidative stress: Mitochondria in stressed or ischaemic tissues produce nitric oxide (mtNO) that binds to cytochrome c oxidase competitively displacing oxygen leading to oxidative stress and reduced ATP production. Light of suitable wavelength, sufficient irradiance and time when applied to injuries is absorbed by cytochrome c oxidase displacing mtNO thereby reducing oxidative stress and increasing ATP production. A cascade of downstream metabolic effects lead to a reduction in inflammatory markers including prostaglandin E2, interleukin 1β and tumor necrosis factor α.
2. Analgesia. LLLT creating a nerve block. Higher irradiance/energy treatments can induce an analgesic effect by disrupting fast axonal transport in small diameter fibres, in particular nociceptors. This temporary (reversible) inhibition of A-delta and C fiber transmission reduces tonic peripheral nociceptive afferent input and facilitates reorganization of the modulation of synaptic connections. Repeated treatments lead to a reduction in central sensitization. (Source#3: Low Level Laser Therapy (LLLT) for Orofacial Pain—Seyyed Amir Seyyedi 1, Pooya Olyaee 2, Zohreh Dalirsani 3, Farnaz Falaki 3 Department of Oral Medicine)

When cells are stressed (Arthritis, Osteoporosis, Sports Injuries), nitric oxide (NO) inhibits oxygen consumption by mitochondrialcytochrome c oxidase. This reduces production of ATP and causes oxidative stress leading to increased inflammation and reduced production of ATP.LLLT displaces nitric Oxide (NO) from cytochrome c oxidase thereby reducing inflammation and restoring ATP production, relieving pain and helping tissues heal more quickly.

The laser beam allows for the use of a carefully selected wavelength, coherently directed to specific tissue/cells, provides energy to selective stimulating processes in living cells; this Can help in increasing blood flow, excite cell activity and intensify inter-cell communications. Laser light has been used to treat joint related musculoskeletal pain caused by illnesses including—Tendonitis (Back Pain, Knee Tendonitis, Hand Tendonitis, Achilles Tendonitis) Tennis Elbow, Carpel-Tunnel, Arthritis, Osteoporosis, Plantar Fasciitis, Tissue Healing From Sports Injuries And Bursitis.

FDA Approved References for the Technology in the US—

Exists in other bigger modalities where patients go to clinics to get treated—Source #4 http://www.multiradiance.com/pain-management and Source#5—http://www.trowbridgechiropodist.co.uk/Low-Level-Laser-Treatment.html And their products are FDA approved to show that LLLT for pain relief from arthritis and joint pain related illness is very safe and viable alternative to NSAIDs (Non-Steroidal Anti Inflammatory Drugs).

NASA: Effect of NASA Light-Emitting Diode Irradiation on Molecular Changes for Wound Healing in Diabetic Mice

Source: Journal of Clinical Laser Medicine & Surgery Volume: 21 Number: 2 Page: 67-74 (DOI: 10.1089/104454703765035484 Author(s): Harry T. Whelan M D; Ellen V. Buchmann B S; Apsara Dhokalia PhD; Mary P. Kane B S; Noel T. Whelan B S; Margaret T. T. Wong-Riley PhD; Janis T. Eells PhD; Lisa J. Gould M D, PhD; Rasha Hammamieh PhD; Rina Das PhD; Marti Jett PhD)

In the field of portable phototherapy devices, United States Published Patent Application Number 2008/0255640, filed L by Kipp et al., discloses a portable phototherapy device for treating skin conditions that is packable and can treat various parts of the body. However, Kipp discloses a device that is rigid, uses an ultraviolet blub, and has only one setting. Kipp does not disclose a flexible and programmable phototherapy device. Also reference able is US Patent application number: 20110144727 Publication date: 2011 Jul. 16 whose claim is one device for all solutions without solving/or helping in any particular malaise—a device used for all body parts.

Similarly, also in the field of portable phototherapy devices, U.S. Pat. No. 6,312,451 B1, filed by Jackson Streeter, and approved in 2001, for a Low level laser therapy apparatus, that treats many conditions, doesn't provide the modality for home self-care for patients to use at their convenience of the home, and is complicated to use and requires a mandatory clinician to treat the patient.

Typically, persons wishing to benefit from phototherapy must go to a spa or a professional health provider/clinic. This is because the phototherapy devices currently available are complicated-to-use and bulky and large. Additionally, until recently, a professional was needed to program and monitor a phototherapy session, because optimal parameters, such as wavelength range, relative distribution of the wavelengths within the range (spectrum), time interval for continuous exposure, time interval between two continuous exposures, time rate of energy delivered, accumulated energy density for exposures, and body component(s) irradiated, were not yet available. Now, many of the optimal parameters are better understood. For example, U.S. Pat. No. 6,524,329, issued to Mellen-Thomas Benedict (the inventor of the present invention), discloses a method of illuminating body components that provides beneficial treatment.

Still, the complexity of the current laser devices to use to treat oneself, the inherent in-efficacy of the device and dependence on another individual for treatments and the huge costs to purchase, has made it prohibitive to enable home self care for patients suffering from joint pains.

Thus, there is a need to provide a pre-programmed portable phototherapy device that is embedded within an orthopedic wrap that is pre-calibrated to treat the specific joint for optimal pain relief, and can be used in the comfort of a patient's home. Furthermore, due to the specific ergonomic fit the accuracy of the LLLT treatment delivery method is enhanced immensely, bringing the clinic to the home and making it incredibly simple to use and treat the hard to reach joint pains.

### SUMMARY OF THE INVENTION

A WEARABLE PORTABLE, PRE-CALIBRATED & HANDS-FREE medical device/s for PAIN RELIEF using a sports/orthopedic brace/wrap (for Wrist, Knee, Elbow, Nexk&Shoulder, Ankle&Foot & Back) embedded with therapeutic Low Level Laser therapy (LLLT) technology based on infrared and near infrared light.

A WEARABLE (for the Wrist, Knee, Back, Neck & Shoulders, Elbow and Ankle&Foot) &PORTABLE (for use anywhere) battery powered, clinical strength device, based on proven 630 nM upto 904 nM LLLT lasers (LLLT-Low Level Laser Tech)/gallium arsenide (GaAs) lasers/LED/VCSEL diodes technology. The unique modality comes with an innovative &unique custom body countered rubber/neoprene/cloth/resin brace like unisex wrap that comes with different sizes (S/M/L/XL) to fit all patients—EMBEDDED with therapeutic laser LED/VCSEL devices PRE-CALIBRATED for the appropriate optimal treatment power settings and duration, which when turned ON ensures delivery of the optimum number of photons required to the affected area, for optimal pain relief and healing.

A convenient WEARABLE device for pain relief from joint related musculoskeletal pain caused by illnesses such as—TENDONITIS (BACK PAIN, KNEE TENDONITIS, HAND TENDONITIS, ACHILLES TENDONITIS) TENNIS ELBOW, CARPEL-TUNNEL, ARTHRITIS, OSTEOPEROSIS, PLANTAR FASCITIS, TISSUE HEALING from SPORTS INJURIES AND BURSITIS, and as an non-invasive alternative to NSAIDs (Non-Steroidal Anti Inflammatory Drugs)

The therapeutic application of low level laser energy, frequently known as low level laser therapy (LLLT), produces beneficial clinical effects in the treatment of musculoskeletal, neurological and soft tissue conditions. LLLT is non-invasive and avoids the potential side effects of drug therapy. More specifically, LLLT delivers photons to targeted tissue, penetrating the layers of skin to reach internal tissues to produce a specific, non-thermal photochemical effect at the cellular level. Jeffrey R. Basford, Laser Therapy: Scientific Basis and Clinical Role, ORTHOPEDICS, May 1993, at 541.

In one embodiment the method includes the patient wearing the easy-to-wear wrap to the specific joint area (a Wrist-Wrap for the wrist, a Back-Brace for the back, a Knee-Brace for the knee, an Elbow-Brace for the elbow, a Neck & Shoulder-Brace for the neck & shoulder and Ankle/Foot-Brace for the ankle &foot). The method includes LLLT laser beam/s bio-stimulating the affected areas (Wrist/Knee/Back/Elbow/Neck-Shoulders/Ankle-Foot), relieving pain caused by joint diseases including TENNIS ELBOW, CARPEL-TUNNEL, ARTHRITIS, OSTEOPEROSIS, PLANTAR FASCITIS, TENDONITIS (BACK PAIN, KNEE TENDONITIS, HAND TENDONITIS, ACHILLES TENDONITIS), TISSUE HEALING from SPORTS INJURIES AND BURSITIS, and simultaneously encouraging tissue healing.

In one embodiment, the apparatus includes a mobile application coupled to a control unit for selecting and controlling laser energy dosage from about 1 joule/treatment point to about 20 joules/treatment point. The apparatus emits laser energy at a wavelength from about 630 nm to about 904 nm, with a mean power output of between about 25 mW to about 500 mW. (Source#7)

The present invention provides the said treatment in a PRE-CALIBRATED method with a simple ON/OFF button—Specifically the various lasers in the wraps are pre-calibrated with specific treatment times, specific to the joint/s being treated, (And is different for each joint area) using specifically required laser wavelength (nM) to also achieve desired penetration depth for optimal treatment, removing the treatment complexity by using the simple ON/OFF button.

The present invention, i.e., the PRE-CALIBRATED orthopedic laser wrap device, due to its body countering modality approach allows patients to treat the HARD-TO-REACH target areas that cause joint pains. And furthermore because of its modality ensures optimal accuracy of the treatments to the affected areas taking away the clinical complexity, makes it simple to use, and brings a clinical-strength solution to the home. Making it a truly differentiated solution, due to the fact that no other such device exists in the marketplace.

The UBER-CONVENIENT, SELF-CARE device allows for the Patient to capture their pain measurement levels using the display on the medical device provided which auto-syncs to the Smartphone app, to historically capture their pain measurements for clinical usage and eventually enable better outcomes for patients.

Inventions:
1. LW-back-1000 Laser-wrap for the human back—For Arthritis, Osteoporosis, etc., and related joint pain relief for the back
2. LW-wrist-1000 Laser-wrap for the human wrist—For Carpel tunnel, etc. for joint pain relief for the wrist
3. LW-Ankle&Foot-1000 Laser-wrap for the human foot/ankle—For plantar fasciitis, etc., and for joint pain relief for the ankle and foot.
4. LW-Elbow-1000 Laser-wrap for the human elbow—For Arthritis, Bursitis, etc., for joint pain relief for the elbow
5. LW-Neck&Shoulder-1000 Laser-wrap for the human neck/shoulder—For joint pain relief for the neck & shoulder
6. LW-Knee-1000 Laser-wrap for the human knee—For Arthritis, Osteoporosis, etc., and related joint pain relief for the knee.

Each device listed above provides Analgesic effects that lasts for 48 hours, significant reduction of inflammation equal to or better than NSAIDs within 2-12 hours, AND healing time of chronic tendinopathies reduced by 70%. Best non-invasive pain-relief solution in industry with ZERO side-effects. (Source#5—http://www.trowbridgechiropodist.co.uk/Low-Level-Laser-Treatment.html)

In one embodiment, the Self-Care treatment is most accurate for optimal pain relief when recommended treatments of 5-10 times a week for a period of 10-20 minutes per session is applied.

Usage: The patient using the laser-wrap like device with multiple laser devices embedded inside—will WEAR/WRAP the specific devices for the specific area and treats by delivering the optimum number of photons required to the affected area:—The user simply turns the power button ON for PRE-CALIBRATED pain relief treatments of specific durations—specific to treatment area. The same button acts as the PAUSE-RESTART button if required. The device shuts-off automatically after the desired pre-determined and calibrated treatment time is achieved. Recommended treatments provide for proven optimal healing and pain-relief for the said area.

This unique modality of ORTHOPEDIC WRAPS combined with LLLT therapy provides for the only PRE-CALIBRATED, HANDS-FREE, WEARABLE & PORTABLE CLINICAL-STRENGTH medical devices for joint pain relief for the Wrist, Elbow, Neck/Shoulders, Knee, Back & Ankle/Foot—providing for SELF-CARE, easy-to-use and ensuring true convenience (use-at-home)—allowing patients to treat themselves immediately, while saving money and time (clinical visits) for the patient.

In summary, it would therefore be highly desirable to enable patients to have an affordable, hands-free, easy to use, clinical strength, pre-calibrated worry-free pain-relief medical device to use at home, enabling self-care and empowering the patient to improve their lives tremendously. This invention is a portable, hands-free &wearable LLLT orthopedic-wrap type apparatus with which “the clinically proven & optimal pre-calibrated laser energy power dosage (25 mW-500 mW)” is administered automatically, for specific durations and treatment depth, to the specific desired area ensuring maximum efficacy through ergonomic fit, and effectively treat the specific joint/s to provide joint-pain relief.

### BRIEF DESCRIPTION OF THE DRAWINGS

FIG. 1A is an sectional view of an OPEN wide belt (1) containing multiple lighted lasers (2) with a Velcro system (3) to hold the belt in place

FIG. 1B is an sectional view of a CLOSED belt (1) containing multiple lighted lasers (2) with a Velcro system (3) holding the belt in place

FIG. 2 is an sectional view of an OPEN wide belt (1) containing multiple lighted lasers (2) with a Velcro system (3) where subject (4) is about to wear the belt

FIG. 3 is a sectional view of a CLOSED belt (1) containing multiple lighted lasers (2) with a Velcro system (3) where subject (4) has strapped the belt in place around his waist (5)

FIG. 4A is an sectional view of an OPEN wide Knee Wrap (6) containing multiple lighted lasers (7) with a Velcro system (8)

FIG. 4B is an sectional view of a CLOSED wide Knee Wrap (6) containing multiple lighted lasers (7) with a Velcro system (8) holding the closed secure fit

FIG. 4C is an sectional view of an OPEN wide Knee Wrap (6) containing multiple lighted lasers (7) with a Velcro system (8) being applied on the Subjects Knee (9)

FIG. 4D is an sectional view of a CLOSED wide Knee Wrap (6) containing multiple lighted lasers (7) with a Velcro system (8) showing the closed secure fit on subjects Knee (9)

FIG. 5A is an sectional view of a CLOSED Ankle Wrap (10) containing multiple lighted lasers (11) with a Velcro system (12) showcasing a secure fit

FIG. 5B is an sectional view of a CLOSED Ankle Wrap (10) containing multiple lighted lasers (11) with a Velcro system (12) showcasing a secure fit around the subjects ankle (13)

FIG. 6A is a sectional view of an OPEN Hand Wrap (14) containing multiple lighted lasers (15) with a Velcro system (16) used to secure the hand wrap (14)

FIG. 6B is a sectional view of a CLOSED Hand Wrap (14) containing multiple lighted lasers (15) with a Velcro system (16) showing the securely closed hand wrap (14)

FIG. 6C is an sectional view of an OPEN Hand Wrap (14) containing multiple lighted lasers (15) with a Velcro system (16) where subject is about to wrap it around the hand (17)

FIG. 6D is an sectional view of a CLOSED Hand Wrap (14) containing multiple lighted lasers (15) with a Velcro system (16) showing the Hand Wrap (14) securely fastened on the subjects' hand (17)

FIG. 7A is an sectional view of an OPEN Neck & Shoulder Brace Wrap (18) containing multiple lighted lasers (19) with a Velcro belt system (20)

FIG. 7B is a sectional view of a CLOSED Neck & Shoulder Brace Wrap (18) containing multiple lighted lasers (19) with a Velcro belt system (20) used to securely wrap the shoulder brace around the subjects shoulders (21)

FIG. 8A is an sectional view of a OPEN Elbow Brace Wrap (22) containing multiple lighted lasers (23) with a Velcro wrap & belt system (24) showcasing the secure fit

FIG. 8B is an sectional view of a CLOSED Elbow Brace Wrap (22) containing multiple lighted lasers (23) with a Velcro wrap & belt system (24) shown being worn securely on subjects elbow (25)

FIG. 9 is the schematic diagram (26) used to power the electric circuit to operate the lasers and other electronics for all the products

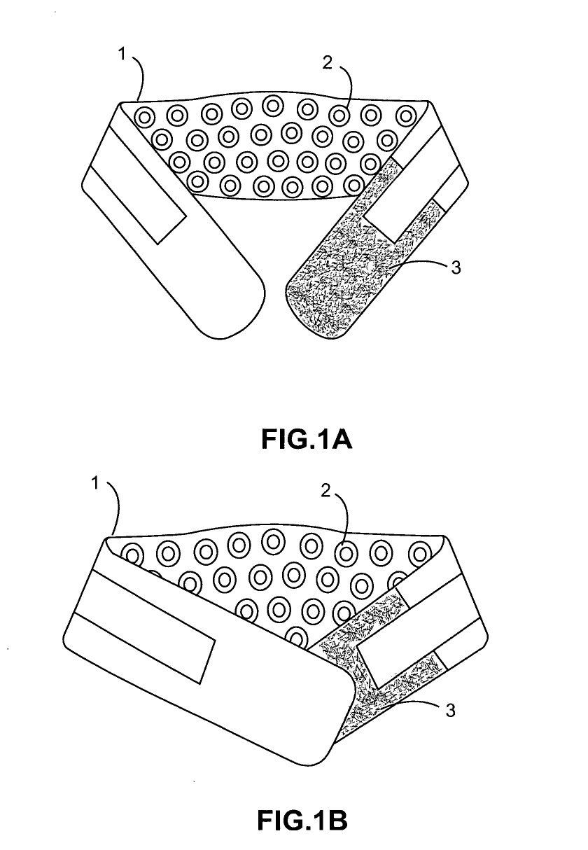

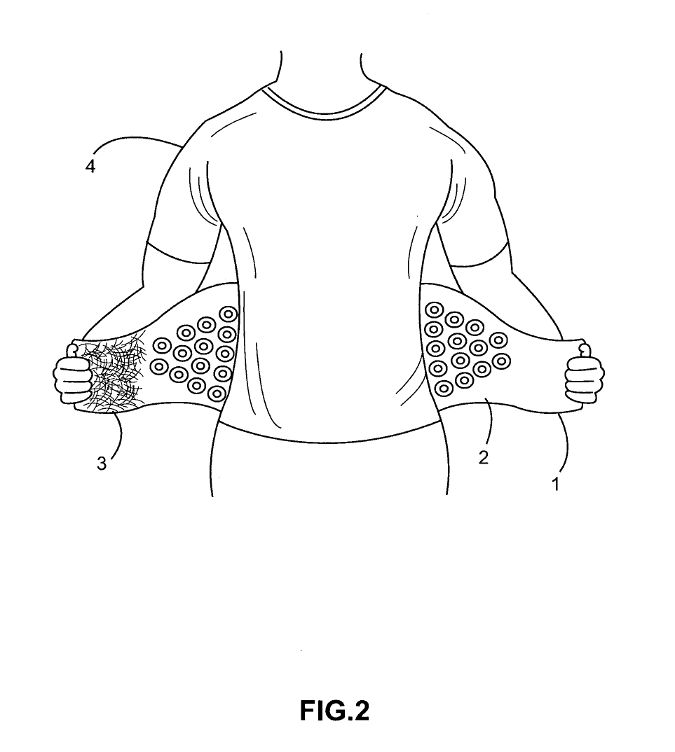

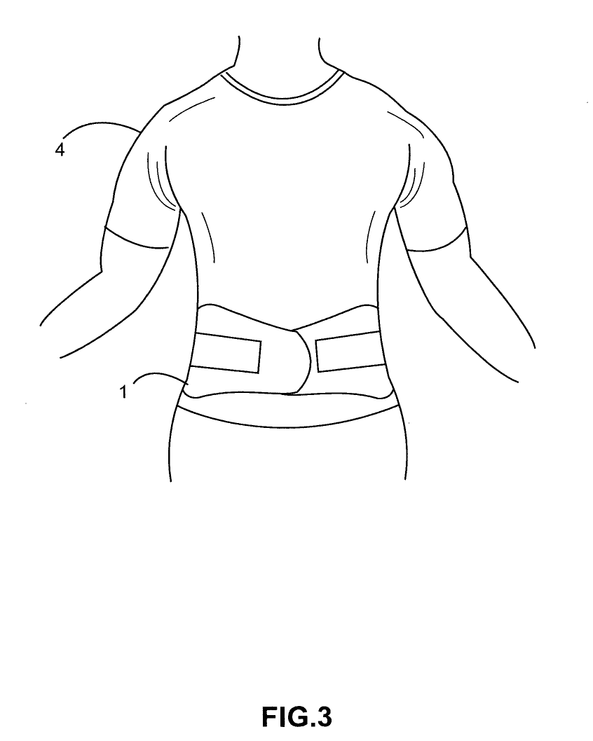

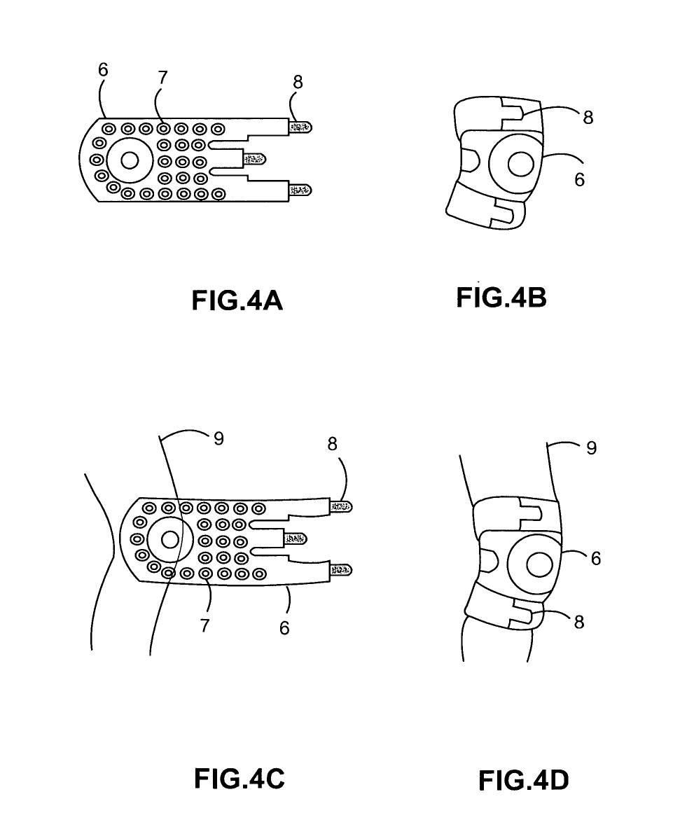

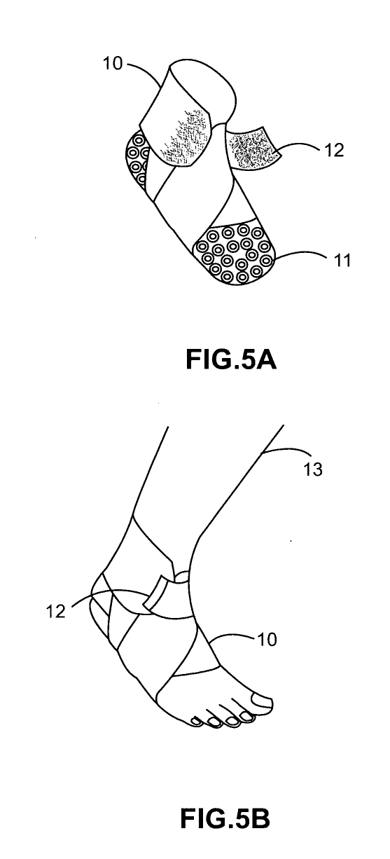

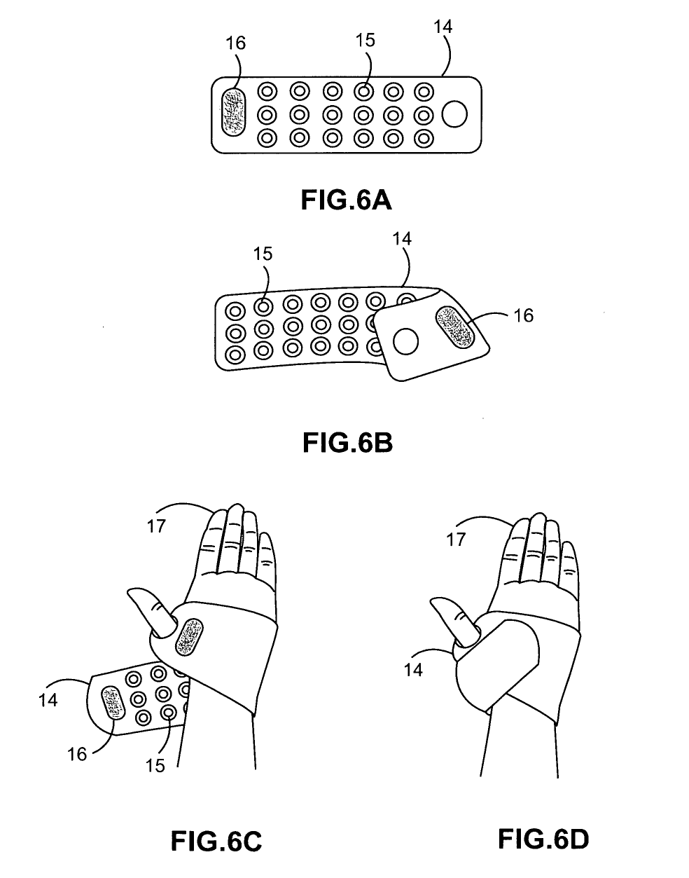

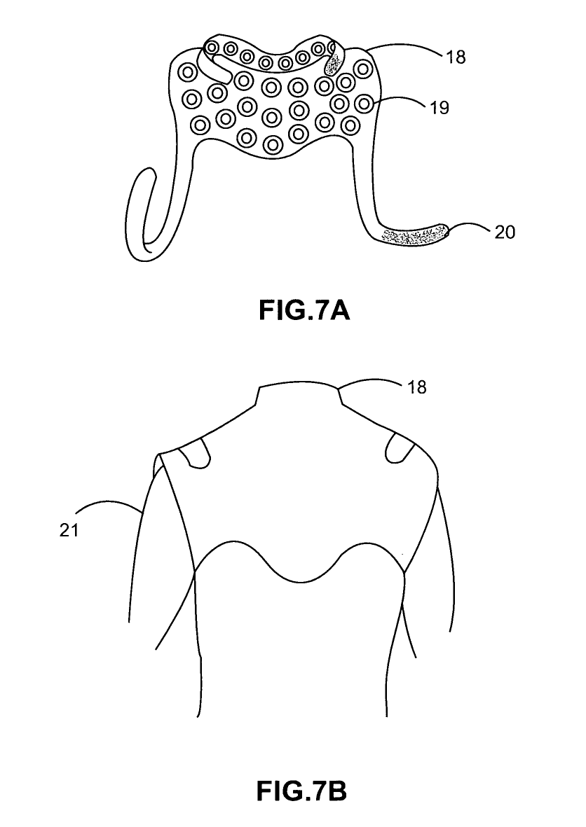

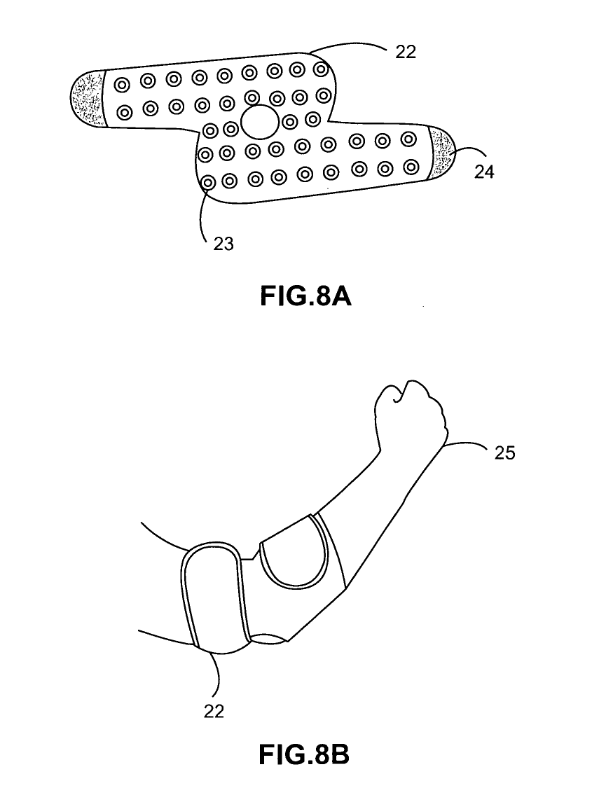

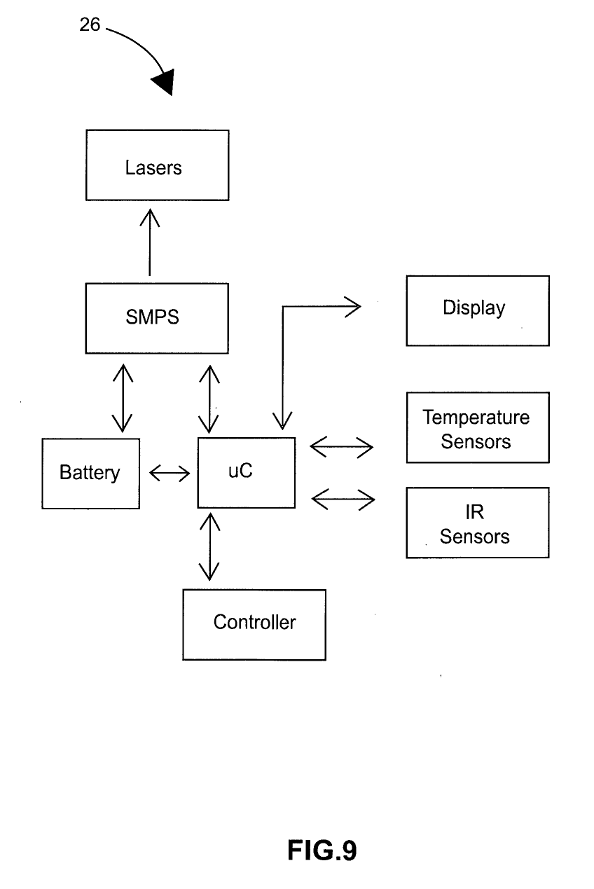

### DETAIL DESCRIPTION OF THE INVENTION

The present invention includes the following Laser Wraps listed below:
LW-back-1000-Laser-wrap (1) for the human back (4)—For Arthritis, Osteoporosis, etc., and related joint pain relief for the back
LW-wrist-1000-Laser-wrap (14) for the human wrist (17)—For Carpel tunnel, etc. for joint pain relief for the wrist
LW-Ankle&Foot-1000 Laser-wrap (10) for the human foot/ankle (13)—For plantar fasciitis, etc., and for joint pain relief for the ankle and foot
LW-Elbow-1000 Laser-wrap (22) for the human elbow (25)—For Arthritis, Bursitis, etc., for joint pain relief for the elbow
LW-Neck&Shoulder-1000 Laser-wrap (18) for the human neck/shoulder (21)—For joint pain relief for the neck & shoulder
LW-Knee-1000-Laser-wrap (6) for the human knee (9)—For Arthritis, Osteoporosis, etc., and related joint pain relief for the knee

In one embodiment, the laser wrap devices are very unique in the delivery method of the solution, where the clinical strength lasers are integrated within the laser orthopedic wrap device, in a unique combination that provides maximum pain-relief efficacy because of Ergonomic orthopedic wrap fits the individual snugly for the specific size, and provides accurate delivery of the pre-determined laser energy to the required areas. The required wavelength provides the necessary depth penetration and combined with the PRE-CALIBRATION treatment times for the specific joint area, makes it the uber efficient home-self-care product for the first time in the pain management industry. The device enables patient to capture pain measurement data through a display for historic beneficial purposes and syncs with the complementary smart phone application to enable numerous use cases benefiting the patient.

Product Usage by Patient—

The patient simply follows these simple steps to achieve pain relief for the specific joint (using specific device), at the comfort of his own home/office or any location he/she prefers—

1. Cleans the specific area (Back/Knee/Wrist/Ankle & Foot/Neck & Shoulder) so skin is conducive to treatment
2. Puts on the specific wrap for the specific treatment area, i.e., Back, Knee, Wrist, Ankle & foot, Neck & shoulder and straps it securely
3. Simply pushes the ON/OFF button
4. Waits for treatment to end automatically. User can pause any time to attend to other things and resume till completion of treatment. Each device has been pre-calibrated to automatically run for desired treatment time for the specific joint for maximum pain relief.

The present invention is a battery powered and can be charged using a 230/110V charger that will be provided. All the parts including the Printed Circuit Board (PCB), the Battery, the display unit, and controller are positioned ergonomically and sewn into the orthopedic wraps to make a sleek design that's comfortable to wear by patient.

The present invention also includes the positioning of the laser diodes in the exact locations required with body countering for the most industry best, effective treatment prescribed by leading orthodontic/chiropractor practicing professionals based on extensive research available on LLLT for pain relief today. These laser diodes are embedded into the orthopedic wraps at these exact locations such that only their lens is visible towards the treated areas.

In one embodiment, microprocessor in the device's is programmed to store patient usage data information for individual patients, and this data is auto-synched to a smartphone application, so that the user can easily monitor previous LLLT dosages and patient progress. For example, the microprocessor has an on-board memory for storing patient information. In one embodiment, a data table is stored in the devices' microprocessor memory and includes an identifying code for each previous treatment (for example the date), and the associated dosage(s), treatment times, codes for treatment locations, and other treatment information from previous treatments. In one embodiment, the patent data information includes a code for specifying the level and location of the patient's pain on each previous treatment, and is captured by the patient using the screen display on the device. The present invention allows the patient to treat him/herself at home, according to the recommended pre-calibrated dosage automatically provided at any time, any place due to its portability and simple automated operation.

This flexible home treatment enables self-care and immediate treatments, improving patient lives while providing for maximum pain relief for the specific related illnesses like osteoporosis and arthritis, Carpel Tunnel, Tennis Elbow, Plantar Fasciitis, and other joint illnesses.

‘The present invention utilizes the LLLT laser light when used in therapy with LLLT 630 nM-904 nM (nanometers) lasers, with power density (irradiance) between 25 mw to 500 mW, generating 1-10 joules/cm2 proven (similar to clinical LLLT products) to effectively reduce inflammation and pain, induce anti-inflammatory cellular activities, induce skin rejuvenation, induce cellular level healing activities.

In another embodiment—Until now, the industry has been using either invasive drugs or large expensive laser machines where patients need to be treated clinically with nurse supervision, causing inconvenience and repetitive expenses. More recent inventions which are all hand-held treatments ask users to treat themselves by purchasing cumbersome self use units, which do not provide for clinical strength accuracy or efficacy, highly inconvenient because of the apparatus ergonomics and the nature of treatment. Consequently current solutions available today are comparatively less effective than the present invention of laser wraps AND do not accurately provide the required treatment power density in joules/cm2, due to

1. The complications inherent in reaching the desired depth to treat affected areas Vs treating just the skin—
2. Due to the inherent nature of handhelds, are not as effective because of the required ergonomics which only our laser wraps can provide, since the user will wear it as a wrap for treatment.
3. Current solutions are complex and most handhelds are inconvenient—& uncomfortable to use due to the hard edges they present, making it less efficient, because it doesn't conform to the body contour—easily missing the target areas, especially when self-treated by patient at home

Furthermore, as an example—since the patient wears ergonomic wraps—the tight fit provides additional joint pain relief similar to the arthritic gloves available in the marketplace today. (http://www.amazon.com/Therall-Arthritis-Gloves/dp/B002QZYILE)

The present invention—‘WEARABLE’ Laser wraps application modality provides for such convenience using laser wraps for the affected areas, with accurate proximity and automated timers for optimal treatment for joint arthritis pain relief, and accelerated healing—making it the most simplest and clinical strength products in the marketplace

Thus it is highly desirable for wearable, light, inexpensive, hands-free portable wearable devices where consumer buys it once and treats himself/herself conveniently and easily by wearing the device, and simply pressing the ON/OFF button, at the comfort of their home or anywhere while going about their daily activities.

The present invention, since its battery operated—is highly portable for use anywhere patient decides, making it extremely convenient, and not having to take invasive drugs, while also avoiding the side effects. It comes with a convenient device holder and DC charger Joules delivered Vs energy density delivered to target area

From Power Density to Dose Now it is possible to understand the number of Joules to be delivered to a target area. Again it is not the total number of Joules delivered at a certain depth that is important since we can see that the beam is spread over an area and we are not treating infinitely small regions. Instead, the important parameter is the energy density; that is, energy per unit area, more commonly called dose with units of J/cm2. From the depth dose profile, a distinct version of which is necessary for each wavelength, frequency, and power setting as well as for every type of material through which the laser beam will penetrate (skin, bone, soft tissue, fat, etc.), you can map out the intensity and determine the power density across a desired area. From the power density at a given depth, we can then revert to the same simple formula used in vitro: power density in units of Watts/cm2 multiplied by treatment time in seconds' yields dose in units of Joules/cm2.
Our Laser wrap devices are already preset for the optimum energy density (i.e. dose) required for the specific area, so user just has to use the ON/OFF switch for treatment, making it the most convenient device for patient use in the industry Energy Density calculation is straightforward: power density in units of Watts/cm2 multiplied by treatment time in seconds yields dose in units of Joules/cm2. This is the energy deposited per area of irradiated tissue.

TABLE 1

| | |
|---|---|
| 1. Coherence | Perfect coherence -Laser Wrap LW-back-1000/LW- |
| | wrist-1000/LW-Ankle&Foot-1000/LW-Knee-1000/LW- |
| | Neck&Shoulder-1000/LW-Elbow-1000 all have a |
| | coherent beam over an area as large as 1.4-4.5 cm² based |
| | on the joint being treated |
| 2. Treatment | A very large area of 4.5 cm² is covered in each treatment |
| area and time | with one completely coherent beam. Due to this high |
| | efficiency, a smaller number of shorter treatments are |
| | required. |
| 3. Peak power | High - up to 200 mW (a half watt) in micro-pulses or |
| | regular, which guarantees maximal penetration and |
| | effectiveness. |
| 4. Energy | High - average 0.9-20 joules per minute per cm² |
| density | |
| 5. Peak | 14.4 joules over the entire treated area. Extremely high |
| energy per | power contributes to greater effectiveness |
| minute | |
| 6. Micro- | Unlike continuous waves, micro-pulses allow the |
| pulses | beam's power and ability to deeply penetrate to the |
| | source of the problem, thus increasing the treatment's |
| | effectiveness |
| 7. Weight | 150-200 g - light, portable and rechargeable - ready to |
| | use anywhere at any given moment |
| 8. Operation | Wear it and turn device on by pressing the ON button. |
| methods | Device automatically shuts-off after predetermined |
| | treatments. Counter displays usage time and No of |
| | usages and other metrics |
| 9. Historic | User can capture pain measurement data on devices. |
| Pain Data | Device auto-synchs via Bluetooth with Smartphone |
| | application provided |
| 10. Safety | No need for safety goggles - Class 1 safety approval |
| goggles | |
| 11. Modality | Laser Wrap LW-Back-1000 Sample specifications |
| | (For Back joint related Pain relief) |
| | Laser Wrap LW-Knee-1000 Sample specifications |
| | (For Knee joint related Pain relief) |
| | Laser Wrap LW-Wrist-1000 Sample specifications |
| | (For Wrist joints related Pain relief) |
| | Laser Wrap LW-Ankle&Foot-1000 Sample specifi- |
| | cations (For Ankle & Foot joints related Pain relief) |
| | Laser Wrap LW-Neck&Shoulder-1000 Sample specifi- |
| | cations (For Neck & Shoulder joints related Pain relief) |
| | Laser Wrap LW-Elbow-1000 Sample specifications |
| | (For Elbow joints related Pain relief) |

1. A portable medical device, comprising:
a plurality of battery powered low level laser therapy (LLLT) laser diodes embedded within a wrap and positionable around an orthopedic joint in a patient, wherein the diodes are able to:
generate a pre-calibrated radiation dosage of a laser-light created by the laser diodes into the joint and at a penetration depth that are recommended by clinicians for treating the joint and/or a clinical disorder;
apply a treatment of the radiation to a portion of at least one of a wrist, a knee, a back, an elbow, a neck & shoulder, and an ankle/foot joint corresponding to the placement of the brace;
reduce a pain and an inflammation caused by at least one of a disease associated with a degradation of a joint or sports related injuries in the at least one of wrist, knee, back, elbow, neck/shoulder, and ankle/foot; and
wherein the medical device comprises the laser diodes embedded within at least one of a glove, a knee brace, a back brace, an elbow-brace, a neck/shoulder-brace, and an ankle/foot brace.

2. The device of claim 1, wherein the laser diodes comprise light emitting (LED), or vertical-cavity surface-emitting laser diodes (VCSEL), pre-calibrated to emit a dosage specific to a joint and/or a type of medical disorder.

3. The device of claim 1, wherein a patient wears the medical device by wrapping the device comfortably to the contours of the joint thereby automatically aligning the laser diodes to the clinician recommended target areas, turning it on and freely moving about their daily business/chores, while concurrently receiving a pre-calibrated and pre-determined treatment dosage.

4. The device of claim 1, wherein the medical device provides for pain relief by acting as a brace, and secondly by providing an anti-inflammatory and an analgesic effect via the embedded laser diodes.

5. The device of claim 1, wherein the medical device further comprises a control mechanism with at least one display unit for displaying one or more of: a timer per session, a comprehensive device treatment time, and a historical treatment data; and a patient input mechanism to capture their pain measurement and record it in memory for historical data purposes for an MD to review if required.

6. The device of claim 1, wherein the medical device auto-syncs via Bluetooth to a complementary Smartphone application provided (IOS/Android) which stores a patient's pain and medication data and that can be accessed remotely by an MDs for historic treatment and usage data.

7. The device of claim 1, wherein the medical device provides for the temporary pain-relief related to musculoskeletal pain diseases including—Tennis-elbow, Carpel-tunnel, Arthritis, Osteoporosis, Plantar-fasciitis, and tissue healing from sports injuries & Bursitis, and Tendonitis of the joints comprising (Back Tendonitis, Knee Tendonitis, Hand & Wrist Tendonitis, and Achilles Tendonitis).

8. The medical device of claim 1, further comprising a plurality of low level laser therapy (LLLT) laser diodes embedded with a laser wrap unit that is positionable around an orthopedic joint in a patient, wherein each laser wrap unit has at least one proximity sensor where the lasers diodes will not turn on unless the wrap unit is securely placed and is within less than 1 cm of a patient's skin.

9. The medical device of claim 1, further comprising a Printed Circuit Board (PCB), a battery, a display unit, a controller, and an ON/OFF button that are positioned ergonomically and sewn into an orthopedic wrap that is comfortable to wear by patient.

10. The medical device of claim 1, further comprising the ability to position of the laser diodes in the exact locations on the joint required for the most effective treatment as prescribed by leading orthodontic/chiropractor MDs and practicing professionals based on extensive research available on LLLT for pain relief, wherein the laser diodes are sewn into the orthopedic wraps at these exact locations with only a plurality of laser lens visible towards the treated areas.

11. The medical device of claim 1, further comprising a sensing unit with an ON/OFF button that has at least one heat sensor able to regulate the temperature of the medical device, and wherein the sensing unit is powered by at least one rechargeable battery.

12. The medical device of claim 1, further comprising an ON/PAUSE button to activate, pause, and resume a preprogrammed clinical strength timed treatment.

13. A portable low level laser therapy (LLLT) method for the treatment of joint pain, comprising:
providing an orthopedic joint brace or wrap with a plurality battery powered laser diodes sewn into the brace;
generating a radiation of a laser-light created by plurality of laser diodes;
applying a treatment of the radiation to a portion of a body part of a patient;
reducing a condition of at least one of pain, swelling, inflammation, limited range of motion, and redness of a joint;
reducing a symptom of a disease of a joint; and
providing an LLLT industry standard relief from the condition when the LLLT treatment is complete.

14. The method of claim 13, wherein the brace further comprises a computerized laser diode control mechanism programmed to control the plurality of laser diodes to deliver light energy or radiation in one or more patterns to provide pain relief from a wrist, a knee, a back, an elbow, a neck & shoulder, an ankle/foot and an Neck & Shoulder joint related illnesses, Arthritis, Osteoporosis, Tendonitis, bursitis, and tissue healing from sports related joint injuries.

15. The method of claim 13, wherein the plurality laser diodes are between 10-30 Light Emitting Diodes, Laser Diodes, or vertical-cavity surface-emitting laser (VCSEL) diodes, capable of delivering between 1 to 20 joules of light energy per square centimeter of skin area during the course of a single treatment session that lasts no longer than an hour to provide pain relief for the said joint.

16. The method of claim 13, wherein upon secure placement of the brace on the patient's joint, the lasers diodes are positioned to provide for the laser light dosage at the required penetration depth according to the LLLT medical industry—Chiropractic & Orthopedic standards, to provide maximum LLLT pain-relief, said dosage comprising about 630 to 904 nanometers light wavelength for treating surface to deep tissue pain, with a power density about 25-500 milliwatts, and generating about 1-20 joules/cm².

17. The method of claim 13, wherein upon secure placement of the brace on the patient's joint, and initiation of therapeutic light treatment, at least some of the plurality of laser diodes will operate, thus exposing at least selected regions of said patient's joint to therapeutic levels of light illumination or irradiation.

18. The method of claim 13, wherein upon secure placement of the brace on the patient's joint the patient chooses up to 3 different laser diode (LED/VCSEL) wavelengths ranging between 630 nm and 904 nm, to treat the patient's joint for optimal treatment depths of the radiation into the joint and durations of treatment times, corresponding to the illness being treated.

19. The method of claim 13, the brace further comprises at least one proximity sensor, at least one heat sensor, and a regulating mechanism, wherein the laser diodes will not be turned on unless the brace is securely placed on the patient joint, within less than 1 cm of the skin area, the at least one heat sensors detect the temperature of the skin area being treated, and the regulating mechanism reduces or terminates laser exposure to at least those regions of the said joint where the detected temperature exceeds a preset threshold.

20. The method of claim 13, the patient experiences joint related pain relief from joint related illnesses including Tennis Elbow, Carpel-Tunnel, Arthritis, Osteoporosis, Plantar Fasciitis, and Tissue healing from sports injuries and Bursitis and Tendonitis of the joints comprising Back Tendonitis, Knee Tendonitis, Hand/Wrist Tendonitis, Achilles Tendonitis.
